(19) United States
(12) Patent Application Publication
(10) Pub. No.: US 2026/0345112 A1
(43) Pub. Date: Nov. 5, 2026

(54) METHOD FOR DETECTING CARDIAC VALVE REGURGITATION AND ULTRASOUND SYSTEM

(71) Applicant: GE Precision Healthcare LLC, Waukesha, WI (US)

(72) Inventors: Gang Liu, Wuxi (CN); Yuanlong Meng, Wuxi (CN); Huan Liu, Wuxi (CN)

(21) Appl. No.: 19/556,864

(22) Filed: Mar. 4, 2026

(30) Foreign Application Priority Data
Mar. 4, 2025 (CN) 202510248476.5

(51) Int. Cl. A61B 8/08; A61B 8/00; A61B 8/06; G06T 5/00; G06T 7/11

(52) U.S. Cl. CPC A61B 8/0883; A61B 8/06; A61B 8/488; A61B 8/5223; A61B 8/5246; G06T 5/00; G06T 7/11; G06T 2207/10132; G06T 2207/20132; G06T 2207/20172; G06T 2207/30048; G06T 2207/30104

(57) ABSTRACT

A method for detecting heart valve regurgitation, including: acquiring a sequence of B-mode ultrasound data to be processed, and a sequence of CF-mode ultrasound data corresponding to the B-mode ultrasound data; separately performing preprocessing targeting a target region on the sequences of the B-mode ultrasound data and the CF-mode ultrasound data; performing secondary processing on the preprocessed sequences of the B-mode ultrasound data and the CF-mode ultrasound data, to acquire a valve closure region; performing regurgitation feature analysis in the valve closure region, to determine a regurgitation frame range; and extracting target data from the sequence of the CF-mode ultrasound data on the basis of the regurgitation frame range, and generating a regurgitation region identifier.

### CROSS-REFERENCE TO RELATED APPLICATION

This application claims priority to Chinese Patent Application No. 202510248476.5, which was file on Mar. 4, 2025 at the Chinese Patent Office. The entire contents of the above-listed application are incorporated by reference herein in their entirety.

### TECHNICAL FIELD

The present disclosure relates to a method for detecting heart valve regurgitation, and an ultrasound system for detecting heart valve regurgitation.

### BACKGROUND

Ultrasound diagnosis of heart valve regurgitation is an important routine examination, and is also a crucial step in the evaluation of cardiovascular disease. Currently, clinical practice primarily relies on two-dimensional ultrasound imaging (B-mode) combined with color flow Doppler (CF-mode) for detection of regurgitation. The B-mode is a fundamental mode of ultrasound imaging, which generates two-dimensional gray-scale images of cardiac structures by means of transmitting ultrasound waves and receiving reflected signals. The CF-mode is a color flow imaging technique based on the Doppler effect, used for real-time display of blood flow direction and velocity within the heart.

During regurgitation detection, a physician needs to manually operate an ultrasound probe to locate the spatial position of a heart valve in the B-mode, then switch to the CF-mode to capture temporal information on regurgitation, and identify a regurgitation event through repeated observation of dynamic changes in blood flow. If further quantitative analysis is required, spectral Doppler can be added, to measure parameters such as regurgitation velocity and pressure gradient. However, this process requires step-by-step operations for spatial positioning and temporal detection, which relies on the physician's experience and is time-consuming. Although automated detection means have been developed for regurgitation detection, real-time processing of a large number of image frames imposes high demands on hardware supporting the algorithm.

### SUMMARY

The present disclosure is made in view of the above problems, and an object thereof is to provide a method and an ultrasound system for detecting heart valve regurgitation, which can simplify a large amount of data required to be processed in conventional detection, thereby reducing computational complexity, and can also achieve automation and precision of the detection process, thereby providing an efficient, robust, and low-cost solution for heart valve regurgitation detection.

A first aspect of the present disclosure relates to a method for detecting heart valve regurgitation, comprising: acquiring a sequence of B-mode ultrasound data to be processed, and a sequence of CF-mode ultrasound data corresponding to the B-mode ultrasound data; separately performing preprocessing targeting a target region on the sequences of the B-mode ultrasound data and the CF-mode ultrasound data; performing secondary processing on the preprocessed sequences of the B-mode ultrasound data and the CF-mode ultrasound data, to acquire a valve closure region; performing regurgitation feature analysis in the valve closure region, to determine a regurgitation frame range; and extracting target data from the sequence of the CF-mode ultrasound data on the basis of the regurgitation frame range, and generating a regurgitation region identifier.

A second aspect of the present disclosure lies in that, in the first aspect, the target region is a region of interest comprising a heart valve, and the preprocessing comprises: performing detection on each frame of data in the sequence of the B-mode ultrasound data using a target detection algorithm, to acquire position coordinates of the region of interest in each frame of data; calculating a unified cropping region on the basis of position coordinates of the region of interest; and separately performing cropping processing on the sequences of the B-mode ultrasound data and the CF-mode ultrasound data according to the unified cropping region.

A third aspect of the present disclosure lies in that, in the first aspect or second aspect, the secondary processing comprises dimensional reconstruction, the dimensional reconstruction comprising unfolding the sequences of the B-mode ultrasound data and the CF-mode ultrasound data from three-dimensional data to two-dimensional data along a time dimension.

A fourth aspect of the present disclosure lies in that, in the third aspect, the secondary processing further comprises feature extraction, the feature extraction comprising: performing trimming on the two-dimensional data for the CF-mode ultrasound data on the basis of blood flow, to obtain a blood flow region range, and performing trimming on the two-dimensional data for the B-mode ultrasound data in the same manner according to the blood flow region range; and analyzing a gray-scale intensity distribution of the trimmed two-dimensional data for the B-mode ultrasound data, to determine the valve closure region.

A fifth aspect of the present disclosure lies in that, in the first aspect or second aspect, the regurgitation feature analysis comprises: identifying regurgitation feature points within the valve closure region; and determining the regurgitation frame range on the basis of distribution characteristics of the regurgitation feature points.

A sixth aspect of the present disclosure lies in that, in the first aspect or second aspect, the target data comprises a plurality of regurgitation frames, and the regurgitation region identifier is generated by means of the following steps: extracting the regurgitation frames from the sequence of the CF-mode ultrasound data within the regurgitation frame range; performing segmentation on the regurgitation frames using a segmentation algorithm, to generate the regurgitation region identifier; and mapping the regurgitation region identifier to a raw ultrasound image.

A seventh aspect of the present disclosure lies in that, in the first aspect or second aspect, the sequence of the B-mode ultrasound data and the sequence of the CF-mode ultrasound data represent time sequences of two-dimensional ultrasound images within a same cardiac cycle, wherein the sequence of the B-mode ultrasound data represents heart valve motion information, and the sequence of the CF-mode ultrasound data represents blood flow information.

An eighth aspect of the present disclosure lies in that, in the first aspect or second aspect, the regurgitation region identifier is a binary regurgitation mask image.

A ninth aspect of the present disclosure lies in that, in the first aspect, the method further comprises performing display enhancement processing on a regurgitation region of an ultrasound image marked by the regurgitation region identifier, the display enhancement processing comprising: reducing image visual characteristics of a non-regurgitation region that is not marked by the regurgitation region identifier; and maintaining velocity attributes of the regurgitation region unchanged.

A tenth aspect of the present disclosure lies in that, in the ninth aspect, the image visual characteristics comprise image brightness and image saturation.

An eleventh aspect of the present disclosure lies in that, in the first aspect, the method further comprises performing highlighted playback processing on regurgitation frames comprised in the target data, the highlighted playback processing comprising automatically reducing a playback speed of the regurgitation frames during playback.

A twelfth aspect of the present disclosure lies in that, in the first aspect, the method further comprises automatic measurement processing, the automatic measurement processing comprising: in a real-time scan mode, automatically positioning a cursor on the basis of the regurgitation region identifier, and automatically initiating an ultrasound measurement mode; and automatically executing measurement of a parameter related to regurgitation.

A thirteenth aspect of the present disclosure lies in that, in the twelfth aspect, the ultrasound measurement mode comprises a continuous wave Doppler mode.

A fourteenth aspect of the present disclosure relates to an ultrasound system for detecting heart valve regurgitation, comprising: a processor, the processor being configured to execute the method according to any one of the first to thirteenth aspects; and an ultrasound probe, the ultrasound probe being controlled by the processor to acquire the sequences of the B-mode ultrasound data and the CF-mode ultrasound data.

A fifteenth aspect of the present disclosure relates to a non-transitory computer-readable storage medium, wherein the non-transitory computer-readable storage medium stores a computer program that, when executed by a processor, implements the method according to any one of the first to thirteenth aspects.

### BRIEF DESCRIPTION OF THE DRAWINGS

The present disclosure can be better understood by means of the description of the exemplary embodiments of the present disclosure with reference to the drawings. In the drawings:

FIG. 1 is a flowchart illustrating a method for detecting heart valve regurgitation according to some implementations of the present disclosure;

FIG. 2 is a schematic diagram illustrating sequences of multi-modal ultrasound data according to the implementation of FIG. 1 of the present disclosure;

FIG. 3 is a flowchart illustrating specific steps of preprocessing according to the implementation of FIG. 1 of the present disclosure;

FIG. 4 is a schematic diagram illustrating a processing procedure of the preprocessing according to the implementation of FIG. 1 of the present disclosure;

FIG. 5 is a schematic diagram illustrating the processing procedure of the preprocessing according to the implementation of FIG. 1 of the present disclosure;

FIG. 6 is a flowchart illustrating steps of secondary processing according to the implementation of FIG. 1 of the present disclosure;

FIG. 7 is a schematic diagram illustrating a processing procedure of the secondary processing according to the implementation of FIG. 1 of the present disclosure;

FIG. 8 is a schematic diagram illustrating a processing procedure of the secondary processing according to the implementation of FIG. 1 of the present disclosure;

FIG. 9 is a flowchart illustrating steps of determining a regurgitation frame range according to the implementation of FIG. 1 of the present disclosure;

FIG. 10 is a schematic diagram illustrating a processing procedure for determining the regurgitation frame range according to the implementation of FIG. 1 of the present disclosure;

FIG. 11 is a flowchart illustrating steps of generating a regurgitation region identifier according to the implementation of FIG. 1 of the present disclosure;

FIG. 12 is a schematic diagram illustrating a processing procedure for generating the regurgitation region identifier according to the implementation of FIG. 1 of the present disclosure;

FIG. 13 is a schematic diagram illustrating an ultrasound image processing result according to some implementations of the present disclosure;

FIG. 14 is a schematic diagram illustrating an ultrasound image processing result according to some other implementations of the present disclosure; and

FIG. 15 is a block diagram illustrating a simplified structure of an ultrasound system according to some implementations of the present disclosure.

### DETAILED DESCRIPTION

Specific implementations of the present disclosure will be described below. It should be noted that in the specific description of said implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present disclosure, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present disclosure belongs. The terms “first” and “second” and similar terms used in the description and claims of the patent application of the present disclosure do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

According to some implementations of the present disclosure, a method for detecting heart valve regurgitation is provided.

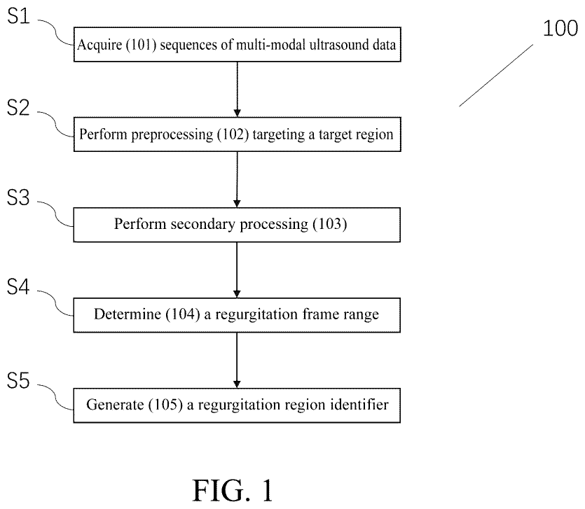

FIG. 1 is a flowchart illustrating a method 100 for detecting heart valve regurgitation according to some implementations of the present disclosure. As shown in FIG. 1, the method 100 for detecting heart valve regurgitation according to the present disclosure includes processing in steps S1 to S5. Steps S1 to S5 are each described below.

Step S1: acquiring (101) sequences of multi-modal ultrasound data.

Specifically, in step S1, a sequence of B-mode ultrasound data and a sequence of CF-mode ultrasound data of a heart (hereinafter sometimes collectively referred to as sequences of multi-modal ultrasound data, or sometimes simply referred to as sequences) are acquired using an ultrasound device. The sequence of the B-mode ultrasound data and the sequence of the CF-mode ultrasound data represent time sequences of two-dimensional ultrasound images within a same cardiac cycle. The sequences are time-continuous frame sequences, containing at least one complete cardiac cycle. The sequence of the B-mode ultrasound data mainly represents heart valve motion information, including open and closed states, as well as motion trajectory, etc., of a valve. The sequence of the CF-mode ultrasound data mainly represents blood flow information in the heart, including blood flow direction, velocity distribution, and dynamic changes in abnormal blood flow (e.g., regurgitation). The sequences of the multi-modal ultrasound data may be acquired by means of various types of ultrasound devices, such as transthoracic echocardiography (TTE), transesophageal echocardiography (TEE), handheld ultrasound probes, etc.

After the sequences of the multi-modal ultrasound data are acquired (101) in step S1, the method proceeds to step S2.

Step S2: performing preprocessing (102) targeting a target region.

Specifically, in step S2, in the sequence of the B-mode ultrasound data and the sequence of the CF-mode ultrasound data acquired (101) in step S1, preprocessing (102) targeting a target region is performed to prepare data for subsequent analysis. The target region here is, for example, a region including a heart valve. However, the target region is not limited thereto, and may be extended to other regions of interest as needed.

After the preprocessing (102) is completed, the method proceeds to step S3.

Step S3: performing secondary processing (103).

Specifically, secondary processing (103) is performed separately on the preprocessed (102) sequences of the B-mode ultrasound data and the CF-mode ultrasound data, to acquire a valve closure region, so as to locate a potential regurgitation region. The valve closure region here includes, but is not limited to, a mitral valve closure region, a tricuspid valve closure region, etc.

After the secondary processing (103) is completed, the method proceeds to step S4.

Step S4: determining (104) a regurgitation frame range.

Specifically, regurgitation feature analysis is performed in the valve closure region, to determine (104) a regurgitation frame range. The regurgitation feature analysis includes, but is not limited to, image feature analysis, hemodynamic parameter analysis, etc. A starting frame and an ending frame of regurgitation are positioned by means of the regurgitation feature analysis, to determine (104) the regurgitation frame range.

After the regurgitation frame range is determined (104), the method proceeds to step S5.

Step S5: generating (105) a regurgitation region identifier.

Specifically, on the basis of the regurgitation frame range determined (104) in step S4, corresponding target data is extracted from the sequence of the CF-mode ultrasound data, and a regurgitation region identifier is generated (105). The target data includes a plurality of regurgitation frames.

The method for detecting heart valve regurgitation according to the present implementation can decouple a complex three-dimensional positioning problem into several relatively simple problems in spatial and temporal dimensions, thereby significantly reducing the amount of data to be processed and lowering the consumption of computational resources. This further significantly improves response speed and processing efficiency, demonstrating remarkable effectiveness and strong robustness for detecting heart valve regurgitation.

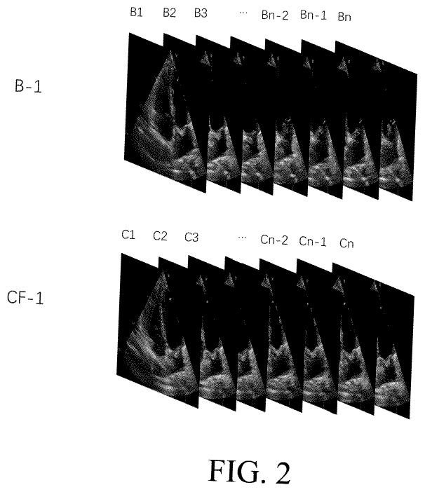

On the basis of FIGS. 2 to 12, the specific implementation of the method is described below in detail, taking mitral valve regurgitation detection as an example.

FIG. 2 is a schematic diagram illustrating the sequences of the multi-modal ultrasound data according to the above implementation of the present disclosure. In step S1, the sequence of the B-mode ultrasound data is acquired (101) using an ultrasound device such as an ultrasound probe. For ease of understanding, an initial state of the acquired sequence of the B-mode ultrasound data is denoted as B-1. As shown in FIG. 2, in the sequence B-1 of the B-mode ultrasound data, frames in the time dimension are B1, B2, B3, . . . , Bn-2, Bn-1, and Bn, respectively, where n represents the number of frames of the sequence in the time dimension, and n is an integer greater than or equal to 1. As described above, in the present implementation, the sequence B-1 of the B-mode ultrasound data is a time-continuous frame sequence, and the time interval between frames is constant. Additionally, the time sequence of the B-mode ultrasound data covers at least one complete cardiac cycle, ensuring that the acquired data can accurately capture a complete dynamic change process of the heart from systole to diastole.

Similarly, in step S1, the sequence of the CF-mode ultrasound data is also acquired (101) using an ultrasound device such as an ultrasound probe. For ease of understanding, an initial state of the acquired sequence of the CF-mode ultrasound data is denoted as CF-1. As shown in FIG. 2, in the sequence CF-1 of the CF-mode ultrasound, frames in the time dimension are C1, C2, C3, . . . , Cn-2, Cn-1, and Cn, respectively, where n represents the number of frames of the sequence in the time dimension, and n is an integer greater than or equal to 1. Similarly, in the present implementation, the sequence CF-1 of the CF-mode ultrasound data is also a time-continuous frame sequence, and is in one-to-one correspondence with the sequence of the B-mode ultrasound data. That is, the sequence of the B-mode ultrasound data and the sequence of the CF-mode ultrasound data represent time sequences of two-dimensional ultrasound images within a same cardiac cycle, and the frames in the sequences correspond one-to-one in the time dimension. After the acquisition (101) is completed, the processing of step S2 is then performed on the acquired sequence B-1 and sequence CF-1.

In the present implementation, in step S2, the preprocessing (102) targeting the target region is performed on the sequence B-1 (B1, B2, B3, . . . , Bn-2, Bn-1, and Bn) and the sequence CF-1 (C1, C2, C3, . . . , Cn-2, Cn-1, and Cn) acquired in step S1. The target region described herein is a region of interest (ROI) including a heart valve. For cardiac ultrasound images, the ROI typically includes the most critical diagnostic or research information, such as heart valves and blood flow channels.

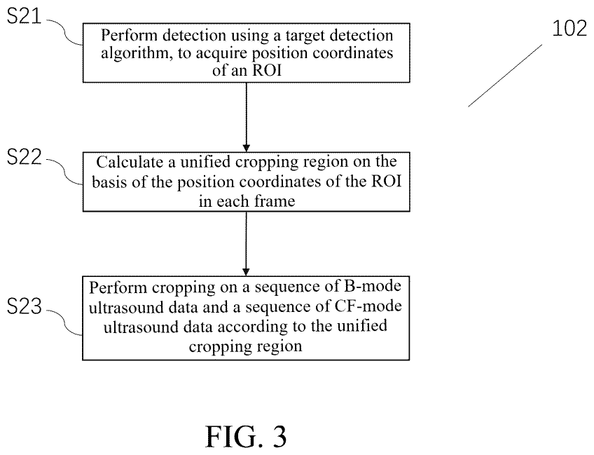

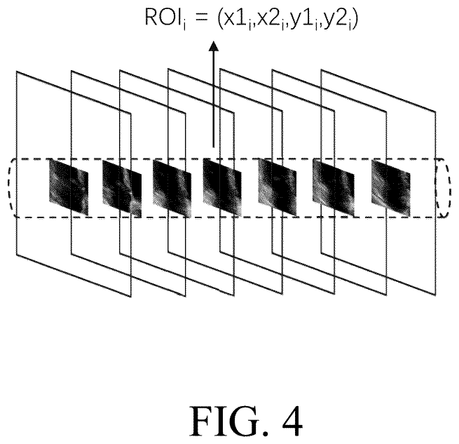

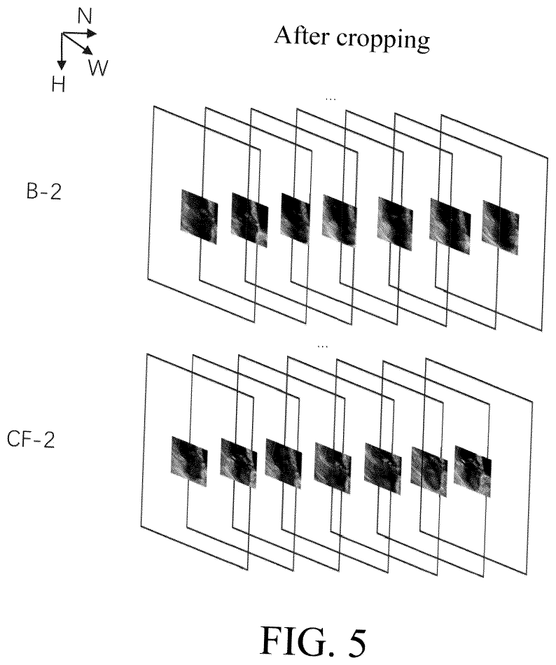

FIG. 3 is a flowchart illustrating specific steps of the preprocessing (102) according to the present implementation. FIGS. 4 and 5 are schematic diagrams illustrating a processing procedure of the preprocessing (102) according to the present implementation. The preprocessing (102) according to the present implementation is described below in detail with reference to FIGS. 3 to 5.

First, in S21, detection is performed on each frame of data (Bi) in the sequence B-1 (B1, B2, B3, . . . , Bn-2, Bn-1, and Bn) of the B-mode ultrasound data using a target detection algorithm, to acquire position coordinates ROIᵢ=(x1ᵢ, x2ᵢ, y1ᵢ, y2ᵢ) of the ROI in each frame of data (as shown in FIG. 4), where ROIᵢ represents the ROI of an i-th frame Bi in B-1, x1ᵢ and x2ᵢ represent the starting coordinates and ending coordinates of the ROI in the horizontal direction (width dimension) in that frame, and y1ᵢ and y2ᵢ represent the starting coordinates and ending coordinates of the ROI in the vertical direction (height dimension) in that frame.

Specifically, the ROI is identified and the position coordinates ROIᵢ are determined by means of analyzing features in the sequence B-1 of the B-mode ultrasound data using the target detection algorithm. In the present implementation, a lightweight backbone target detection algorithm, i.e., a Faster R-CNN algorithm, is used for the target detection algorithm, but the present disclosure is not limited thereto. In some implementations, other target detection algorithms commonly used in the art, such as YOLO, SSD, Mask R-CNN, etc., may also be adopted according to different real-time requirements and accuracy requirements. After the ROIᵢ are acquired, the method proceeds to step S22.

Next, in step S22, a unified cropping region Crop ROI is calculated on the basis of the position coordinates of the ROIᵢ in each frame. In the present implementation, the position coordinates of the unified cropping region are calculated by Crop ROI=(x1, x2, y1, y2), where

x1=x1ᵢ.min(), x2=x2ᵢ.max();

and

y1=y1ᵢ.min(), y2=y2ᵢmax().

According to the above formulas, the values of x1, x2, y1, and y2 are the minimum value of x1ᵢ, the maximum value of x2ᵢ, the minimum value of y1ᵢ, and the maximum value of y2ᵢ in each frame Bi, respectively. In this way, it can be ensured that the finally obtained cropping region Crop ROI can include the ROIs of all frames. However, the method for calculating the cropping region Crop ROI is not limited thereto. In some implementations, the cropping region may be calculated by using a median, a quantile of a certain percentage, a weighted average, or the like. Alternatively, in some implementations, additional constraints may be introduced as needed for adaptive cropping region calculation. Alternatively, in some implementations, a deep learning model may also be utilized to automatically predict and determine the cropping region. After the unified cropping region Crop ROI is calculated, the method proceeds to step S23.

Next, in step S23, the sequence B-1 of the B-mode ultrasound data is cropped according to the unified cropping region Crop ROI. As shown in FIG. 5, for ease of understanding, the cropped sequence of the B-mode ultrasound data is denoted as B-2. Therefore, after cropping is performed according to the position coordinates of the unified cropping region, the dimensions of the sequence B-2 become (N, H, W, 1), where N represents the number of frames in the time dimension, H represents the height of each frame after cropping, W represents the width of each frame after cropping, and 1 represents the number of channels per pixel (gray-scale image).

Next, the sequence CF-1 of the CF-mode ultrasound data is similarly cropped according to the unified cropping region Crop ROI, and the resulting cropped sequence of the CF-mode ultrasound data is denoted as CF-2. As shown in FIG. 5, the dimensions of CF-2 become (N, H, W, 3), where N represents the number of frames in the time dimension, H represents the height of each frame after cropping, W represents the width of each frame after cropping, and 3 represents the number of channels per pixel (color image). At this point, the preprocessing (102) in step S2 is completed, and the method then proceeds to step S3 for the secondary processing (103).

In the present implementation, by means of performing the preprocessing (102), irrelevant or redundant information in the ultrasound images can be removed, and the focus can be concentrated on the region of interest (ROI), thereby improving the efficiency of subsequent processing and enhancing the accuracy of diagnosis. At the same time, by means of cropping the raw data, the amount of data that needs to be processed subsequently can be significantly reduced, the consumption of computational resources can be lowered, and consequently, the response speed and processing efficiency can be improved.

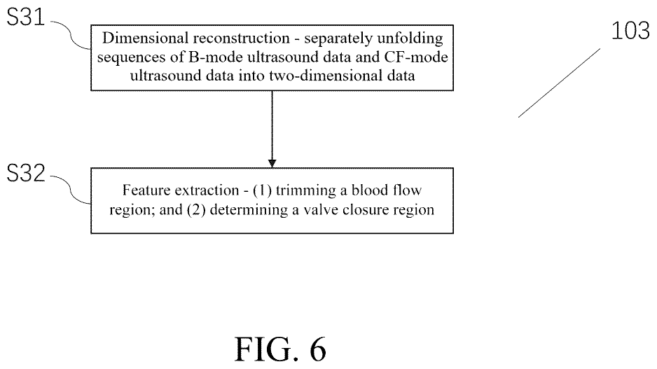

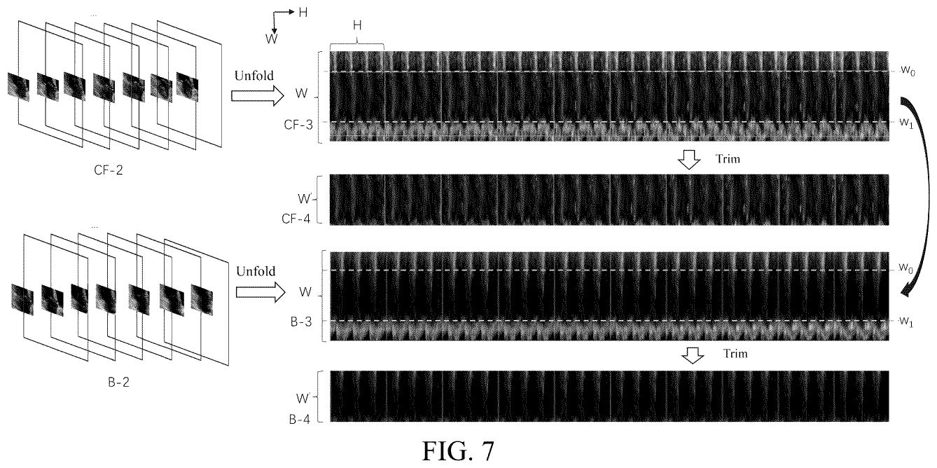

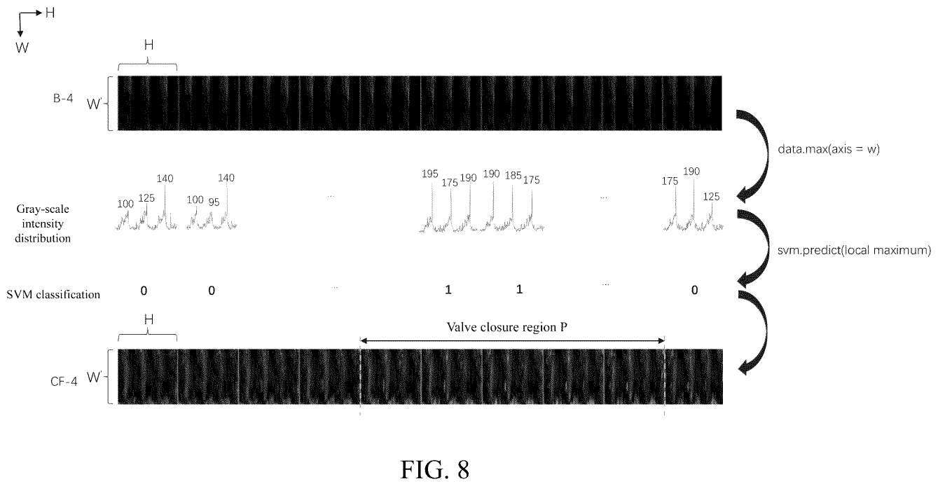

FIG. 6 is a flowchart illustrating steps of the secondary processing (103) according to the present implementation. FIGS. 7 and 8 are schematic diagrams illustrating a processing procedure of the secondary processing (103) according to the present implementation. The secondary processing (103) according to the present implementation is described below in detail with reference to FIGS. 6 to 8.

First, in S31, dimensional reconstruction processing is performed, which includes unfolding the sequence B-2 of the B-mode ultrasound data and the sequence CF-2 of the CF-mode ultrasound data, which have been subjected to the preprocessing (102), from three-dimensional data to two-dimensional data (hereinafter sometimes referred to as two-dimensional images) along the time dimension.

Specifically, first, the sequence CF-2 of the CF-mode ultrasound data is unfolded along the time dimension; the upper half of FIG. 7 shows unfolded two-dimensional data CF-3. As shown in FIG. 7, CF-3 is a two-dimensional image obtained by unfolding and merging each frame of the sequence CF-2 along the time dimension (N). The image width of CF-3 is W, and the image height for each frame in the time dimension (N) is H. Thus, the dimensions of the two-dimensional data CF-3 obtained by unfolding the sequence CF-2 with original dimensions of (N, H, W, 3) along the time dimension (N) become (W, H*N, 3).

Next, the sequence B-2 of the B-mode ultrasound data is unfolded in the same manner; the lower half of FIG. 7 shows unfolded two-dimensional data B-3. As shown in FIG. 7, B-3 is a two-dimensional image obtained by unfolding and merging each frame of the sequence B-2 along the time dimension (N). The image width of B-3 is W, and the image height for each frame in the time dimension (N) is H. Thus, the dimensions of the two-dimensional data B-3 obtained by unfolding the sequence B-2 with original dimensions of (N, H, W, 1) along the time dimension (N) become (W, H*N, 1).

In the present implementation, it is shown that the sequence CF-2 of the CF-mode ultrasound data is subjected to dimensional reconstruction first, and then the sequence B-2 of the B-mode ultrasound data is subjected to dimensional reconstruction. However, the present disclosure is not limited thereto, and there is no particular restriction on the order of performing dimensional reconstruction on the sequence B-2 of the B-mode ultrasound data and the sequence CF-2 of the CF-mode ultrasound data. In some implementations, dimensional reconstruction may be performed on the sequence B-2 of the B-mode ultrasound data first, and then on the sequence CF-2 of the CF-mode ultrasound data. Alternatively, in some implementations, dimensional reconstruction on the two sequences may be performed synchronously.

Next, in S32, feature extraction is performed on the unfolded two-dimensional data B-3 for the B-mode ultrasound data, and the unfolded two-dimensional data CF-3 for the CF-mode ultrasound data, respectively. Specifically, in the present implementation, as shown in FIG. 7, for the CF-mode ultrasound data, on the basis of blood flow intensity, a blood flow region range is trimmed from CF-3 along the width dimension (W), i.e., a portion from w0 to w1 as shown in the figure. The two-dimensional image within the trimmed blood flow region range is denoted as CF-4. The width of the two-dimensional image CF-4 is W′ (w0:w1). Then, on the basis of the blood flow region range, for the B-mode ultrasound data, a corresponding portion in B-3 is trimmed along the width dimension (W) in the same manner. As shown in FIG. 7, the trimmed two-dimensional image within the same range is denoted as B-4. Similarly, the width of the two-dimensional image B-4 is also W′ (w0:w1).

Subsequently, in step S32, first, a gray-scale intensity distribution of the trimmed two-dimensional data B-4 for the B-mode ultrasound data is analyzed, to determine the valve closure region. As is well known, the heart valve should be fully closed under normal conditions, to prevent blood regurgitation. Regurgitation means that blood cannot be effectively blocked by the valve, thus resulting in reverse flow. Therefore, to detect regurgitation points, it is first necessary to evaluate whether the heart valve is fully closed. In an ultrasound image, valve closure and opening lead to different changes in gray-scale intensity. When the valve is closed, the reflection and scattering characteristics of ultrasound waves change, thereby affecting the gray-scale values of the image. Typically, when the valve is closed, reflected sound waves cause there to be higher gray-scale intensity in the image. Moreover, the acoustic characteristics of the valve and the surrounding tissues thereof differ in closed and open states. In the closed state, the structure of the valve is tighter, which may also lead to higher local gray-scale intensity. Therefore, in the present implementation of the present disclosure, in order to evaluate the closed state of the valve, gray-scale intensity analysis is first performed on the two-dimensional data B-4 for the B-mode ultrasound data.

Specifically, as shown in FIG. 8, in the present implementation, for the two-dimensional data B-4 regarding the B-mode ultrasound data, local maximum gray-scale intensities are extracted along the width dimension (W) according to data. max(axis=w). The second row in FIG. 8 shows the gray-scale intensity distribution extracted in this manner. As shown in FIG. 8, the extracted local maximum gray-scale intensity values (numbers shown in the figure) are exemplarily presented in the gray-scale intensity distribution. As can be seen from the figure, for example, the frames included in the two-dimensional data corresponding to the local maximum gray-scale intensity values “195, 175, 190, 190, 185, 175” have local maximum gray-scale intensities. This implies that, in these data frames, the heart valve is highly likely to be in a closed state.

Subsequently, the extracted local maximum gray-scale intensity values are taken as classification features, and a support vector machine (SVM) model is constructed to perform classification prediction, so as to achieve accurate classification of the valve closed state. In the present implementation, the classification prediction is performed according to svm. predict(local maximum), and the classification label is defined as: 0 indicates the valve open state, and 1 indicates the valve closed state. The third row in FIG. 8 shows a result obtained by means of classification prediction using the SVM. As shown in FIG. 8, the portion of data frames having local maximum gray-scale intensity values of “195, 175, 190, 190, 185, 175” is classified as 1, i.e., the valve closed state.

Subsequently, on the basis of the result, a valve closure region P is positioned in the two-dimensional data CF-4. At this point, the secondary processing (103) is completed.

In the present implementation, regarding a threshold used for classification prediction, a method for setting same can be flexibly determined as needed, including, but not limited to, statistical analysis of training data, determination of an optimal threshold using an ROC curve, and model optimization in consideration of the distribution characteristics of gray-scale intensity in combination with medical expertise.

Additionally, an example in which the local maximum gray-scale intensity is extracted along the width dimension (W) is shown in the present implementation, but the present disclosure is not limited thereto. In some implementations, the same can be extended to multi-dimensional gray-scale intensity analysis. Moreover, in some implementations, depending on the target features to be detected, features other than the local maximum gray-scale intensity may be selected, or analysis concerning other features may be added on the basis of the local maximum gray-scale intensity.

Additionally, in the present implementation, an example in which classification prediction is performed using a support vector machine (SVM) model is shown, but the present disclosure is not limited thereto. In some implementations, other classification prediction techniques known in the art may be used, such as deep neural networks, ensemble learning models, and multi-classifier fusion methods.

In the present implementation, by means of performing dimensional reconstruction in the secondary processing (103), i.e., unfolding the sequence B-2 of the B-mode ultrasound data and the sequence CF-2 of the CF-mode ultrasound data from three-dimensional data to two-dimensional data along the time dimension (N), the complexity of the data can be reduced, thereby significantly lowering the computational difficulty and increasing the speed of subsequent processing.

Additionally, in the present implementation, an example is shown in which feature extraction is performed in the secondary processing (103), the blood flow region is trimmed using the CF-mode ultrasound data, the B-mode ultrasound data is trimmed according to the blood flow region range, then the gray-scale intensity distribution is analyzed using the B-mode ultrasound data, and the valve closure region is determined in the CF-mode ultrasound data according to the gray-scale intensity distribution. This approach can make full use of and combine the features of the B-mode ultrasound data and the CF-mode ultrasound data, and can accurately identify the valve closure region through cross-validation and feature extraction, thereby significantly improving the accuracy of detection.

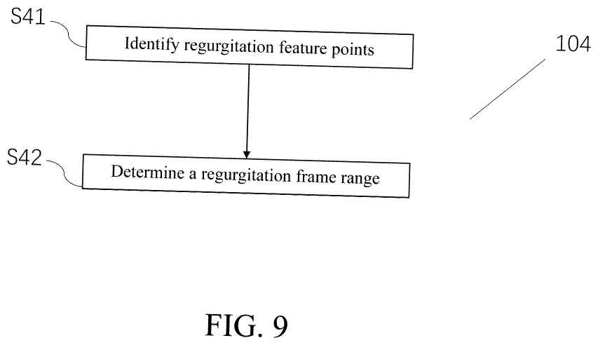

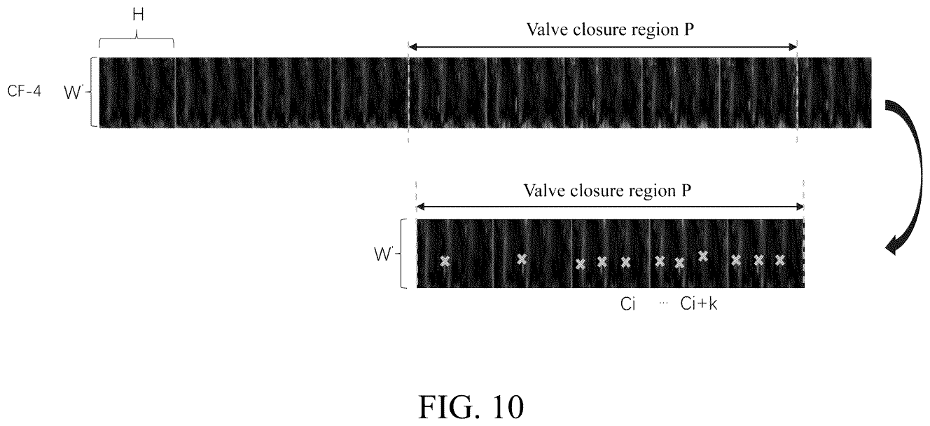

FIG. 9 is a flowchart illustrating steps of determining (104) a regurgitation frame range according to the present implementation. FIG. 10 is a schematic diagram illustrating a processing procedure for determining (104) the regurgitation frame range according to the present implementation. A specific procedure for determining (104) the regurgitation frame range according to the present implementation is described below in detail with reference to FIGS. 9 and 10.

First, in step S41, for the CF-mode ultrasound data, regurgitation feature points are identified within the valve closure region P. Subsequently, in step S42, a regurgitation frame range is determined on the basis of distribution characteristics of the detected regurgitation feature points.

Specifically, as shown in FIG. 10, in the present implementation, the regurgitation feature points are detected for the CF-mode ultrasound data. In FIG. 10, x's annotated in the valve closure region P are the detected regurgitation feature points. Subsequently, cluster analysis is performed on the detected regurgitation feature points, to obtain a regurgitation frame range (Ci . . . Ci+k) along the time dimension (N) in the CF-mode ultrasound data, where Ci represents the starting frame of the regurgitation frame range including the regurgitation frames in the sequence CF-1 of the CF-mode ultrasound data, and Ci+k represents the ending frame of the regurgitation frame range including the regurgitation frames in the sequence of the CF-mode ultrasound data. At this point, processing for determining (104) the regurgitation frame range is completed.

In the present implementation, an example in which the regurgitation feature points are detected and cluster analysis is performed on the regurgitation feature points is shown. The approach described above can accurately locate the positions of the regurgitation feature points by extracting energy using a high-resolution feature thereof. Additionally, by means of performing cluster analysis on the detected regurgitation feature points, the regurgitation frame range can be accurately identified, thereby greatly improving the efficiency and accuracy of subsequent processing.

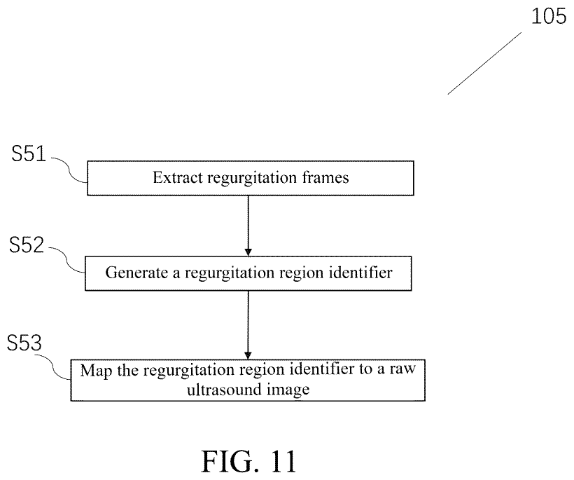

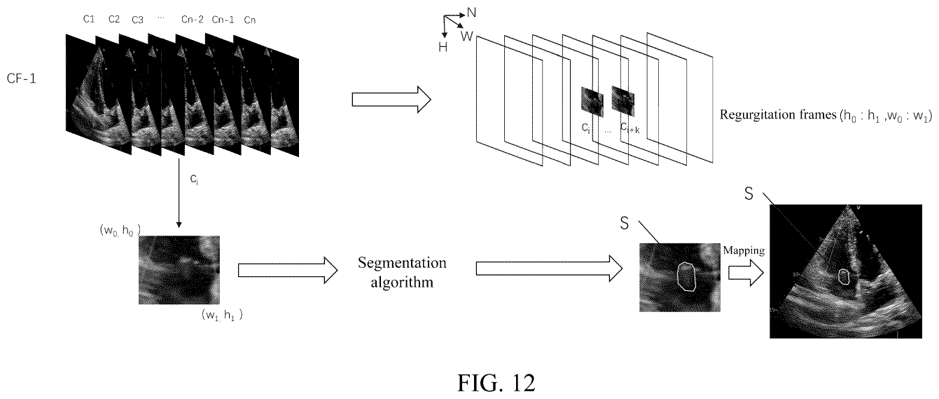

FIG. 11 is a flowchart illustrating steps of generating (105) a regurgitation region identifier according to the present implementation. FIG. 12 is a schematic diagram illustrating a processing procedure for generating (105) the regurgitation region identifier according to the present implementation.

First, in step S51, the regurgitation frames Ci . . . Ci+k are extracted from the sequence CF-1 of the CF-mode ultrasound data on the basis of the regurgitation frame range (Ci . . . Ci+k). Specifically, after the regurgitation frames Ci . . . Ci+k are determined, data of the regurgitation frames Ci . . . Ci+k is extracted in consideration of the position coordinates of the ROI and the blood flow region. For example, as shown in the upper half of FIG. 12, the position coordinates of the extracted regurgitation frames Ci . . . Ci+k are (h0:h1, w0:w1) respectively, where h0 and h1 represent the heights of the position coordinates and w0 and w1 represent the widths of the position coordinates.

Next, the subsequent processing is described using a case in which the regurgitation frame Ci is extracted by means of the described approach as an example. As shown in the lower half of FIG. 12, in step S52, the extracted regurgitation frame Ci is segmented using a segmentation algorithm, to generate a regurgitation region identifier S. The segmentation algorithm includes an encoder path and a decoder path, the encoder path extracting image features by means of convolutional layers and pooling layers, and the decoder path restoring the spatial resolution of a feature map by means of upsampling and deconvolution, and preserving detail information by means of skip connections. Next, in step S53, the regurgitation region identifier S is mapped to a raw ultrasound image. In the present implementation, as shown in FIG. 12, the finally outputted regurgitation region identifier S is a binary regurgitation mask image, in which a pixel value of 1 represents a regurgitation region and a pixel value of 0 represents a non-regurgitation region. At this point, processing for generating (105) the regurgitation region identifier is completed.

In the present implementation, an example in which the extracted regurgitation frame Ci is processed using the segmentation algorithm and the binary regurgitation mask image is generated is shown. The segmentation algorithm may be arbitrary, for example, may be a deep learning-based segmentation algorithm, including, but not limited to, an FCN (Fully Convolutional Network), SegNet, DeepLab series, PSPNet (Pyramid Scene Parsing Network), etc. Additionally, the representation of the regurgitation region identifier S is not limited to a binary regurgitation mask image. In some implementations, multi-value masks, probability maps, contour annotations, or other forms may also be adopted. In a specific implementation, an appropriate segmentation algorithm and representation of the regurgitation region identifier may be selected according to an actual application scenario and performance requirements.

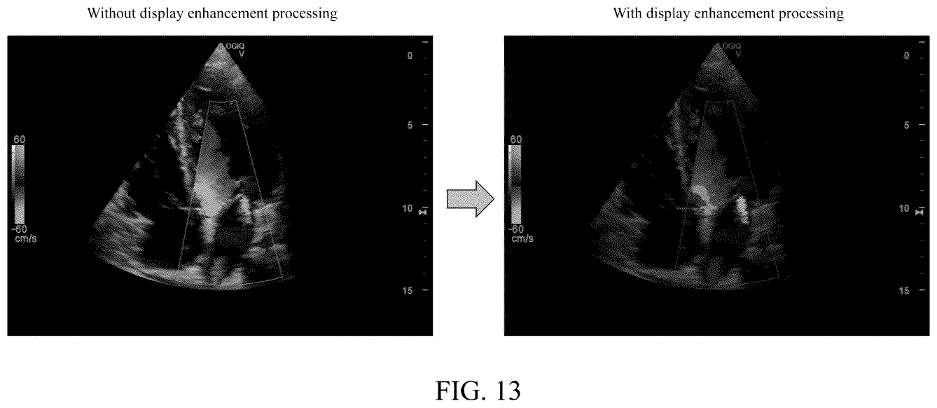

In some implementations of the present disclosure, performing display enhancement processing on the regurgitation region when the regurgitation region identifier S is mapped to the raw ultrasound image is further included. A detailed description is provided below with reference to FIG. 13. FIG. 13 is a schematic diagram illustrating an ultrasound image processing result according to some implementations of the present disclosure.

Specifically, when the regurgitation region identifier S is mapped to the raw ultrasound image as shown in FIG. 12, the display enhancement processing of the regurgitation region is implemented by means of reducing image visual characteristics of a region other than the regurgitation region, i.e., a non-regurgitation region, so as to improve the visualization effect and clinical readability of the regurgitation region. In this way, the features of the regurgitation region can be highlighted, so that the regurgitation region is more prominent in the overall ultrasound image. FIG. 13 shows a processing effect of automatically reducing the brightness and saturation of the non-regurgitation region. The left side of FIG. 13 shows an ultrasound image on which display enhancement processing has not been performed; and the right side shows an ultrasound image on which display enhancement processing has been performed, in which the brightness and saturation of the non-regurgitation region are automatically reduced. Obviously, after the display enhancement processing is performed, the features of the regurgitation region are more obvious, and clinicians can identify regurgitation points more clearly.

In the present implementation, an example in which the image visual characteristics are the image brightness and the image saturation is shown. However, the present disclosure is not limited thereto, and any other parameter may be used as long as a display enhancement effect for the regurgitation region can be obtained. For example, in some implementations, the regurgitation region may be made visually more prominent by means of reducing the contrast of the non-regurgitation region. Alternatively, in some implementations, blurring processing may be further applied to the non-regurgitation region, so as to reduce visual interference of the non-regurgitation region, highlight the regurgitation region, etc.

Additionally, the display enhancement processing according to the implementation of the present disclosure further includes maintaining velocity attributes of the regurgitation region unchanged. In this way, it can be ensured that dynamic characteristics (e.g., blood flow velocity and flow direction) of the regurgitation region can be accurately retained when display enhancement is performed, thereby providing a more reliable diagnostic basis for a clinician.

According to the method for detecting heart valve regurgitation in the above implementation, by means of changing an image presentation mode of the non-regurgitation region and maintaining the velocity attributes of the regurgitation region unchanged, not only can the visualization effect of the regurgitation region be enhanced, but accurate retention of the velocity attributes of the regurgitation region can also be ensured, thereby providing a more intuitive and reliable diagnostic basis for a clinician.

In some other implementations of the present disclosure, performing highlighted playback processing on the regurgitation frames is further included. The implementation of the present disclosure is described below in detail.

Specifically, for a regurgitation frame included in the target data extracted from the sequence CF-1 of the CF-mode ultrasound data, a cinematic slow-motion-like effect is achieved by automatically reducing a playback speed of the regurgitation frame during playback. Such automatic reduction may be performed in a manner known in the art, and may be flexibly selected according to actual requirements. For example, in some implementations, a gradual manner may be employed during velocity changes, to avoid sudden changes that cause discomfort to clinicians. Alternatively, in some implementations, the most suitable playback speed may be automatically adjusted according to the regurgitation features.

According to the method for detecting heart valve regurgitation in the above implementation, when a clinician reviews a stored ultrasound image, regurgitation frames can be automatically recognized and the playback speed can be reduced, so that the clinician does not need to manually adjust the playback speed, thereby significantly reducing operation complexity; and a more intuitive and easy-to-understand visualization effect can be provided, so that even a junior doctor can accurately observe a regurgitation phenomenon, thereby reducing the risk of missed diagnosis and misdiagnosis.

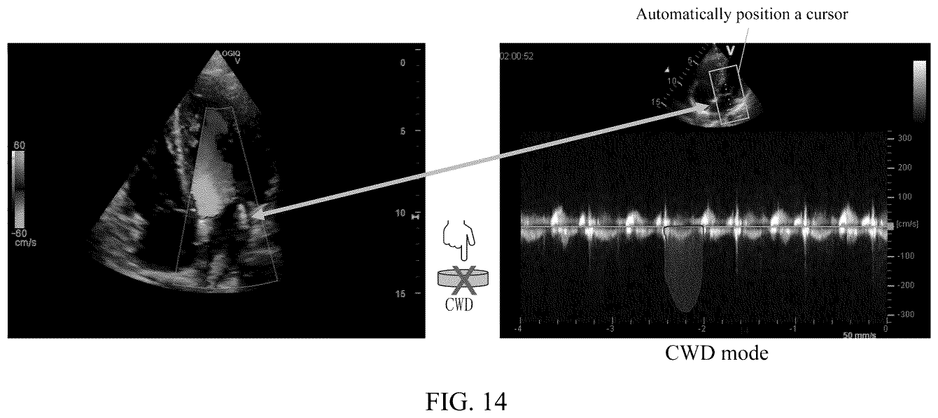

In some other implementations according to the present disclosure, automatic measurement processing is further included. FIG. 14 is a schematic diagram illustrating an ultrasound image processing result according to some other implementations. A detailed description is provided below on the basis of FIG. 14.

The automatic measurement processing according to the present implementation includes: in a real-time scan mode, automatically positioning a cursor on the basis of the regurgitation region identifier S, and automatically initiating an ultrasound measurement mode. Specifically, in the present implementation, in the real-time scan mode, when a regurgitation phenomenon is detected, the cursor is automatically accurately positioned at the regurgitation region, and mode switching is automatically triggered at this time. As shown in FIG. 14, a continuous wave Doppler (CWD) mode is automatically initiated without human intervention. The continuous wave Doppler (CWD) mode is an ultrasound measurement mode in which ultrasound waves are continuously transmitted and received by a dedicated transducer, and blood flow velocity is measured on the basis of the Doppler effect, so that a high-speed blood flow signal can be accurately detected and evaluated. In the present implementation, the ultrasound measurement mode being the continuous wave Doppler mode is shown. However, the present disclosure is not limited thereto, and a suitable ultrasound measurement mode, such as a pulse wave Doppler (PWD) mode, may be selected according to regurgitation features and measurement requirements.

Additionally, automatic measurement processing according to the present implementation further includes automatically performing parameter measurement regarding regurgitation. For example, measurements of parameters such as regurgitation velocity, regurgitation time, regurgitation area, and pressure gradient are performed automatically.

According to the method for detecting heart valve regurgitation in the present implementation, automatic positioning of the cursor and intelligent switching of the measurement mode can be completed without manual operation, and the measurement of regurgitation-related parameters is automatically performed, which significantly improves the examination efficiency and reduces operation steps. Moreover, since all operations can be performed automatically, dependence on the technique of operators is reduced, and the accuracy and consistency of measured positions are ensured.

As described above, the method for detecting heart valve regurgitation according to the above implementation can simplify a large amount of data required to be processed in conventional detection, reduce computational complexity, and can also achieve automation and precision of the detection process, thereby providing an efficient, robust, and low-cost solution for heart valve regurgitation detection.

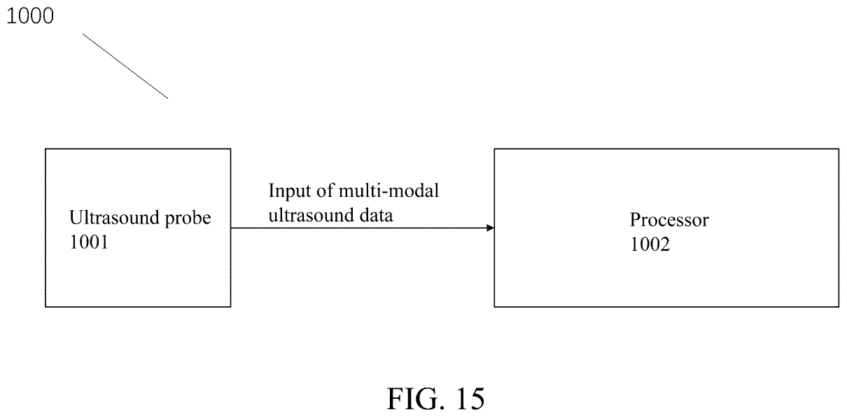

Furthermore, some implementations of the present disclosure further relate to an ultrasound system for detecting heart valve regurgitation. FIG. 15 is a block diagram illustrating a simplified structure of an ultrasound system 1000 according to some implementations of the present disclosure. As shown in FIG. 15, the ultrasound system 1000 includes a processor 1001 and an ultrasound probe 1002. The processor 1001 is configured to execute the method for detecting heart valve regurgitation according to Implementation 1. The ultrasound probe 1002 is controlled by the processor 1001 to acquire sequences of multi-modal ultrasound data, such as a sequence B-1 of B-mode ultrasound data and a sequence CF-1 of CF-mode ultrasound data.

The ultrasound system according to some implementations of the present disclosure can simplify the large amount of data required to be processed in conventional detection, reduce computational complexity, and can also achieve automation and precision of the detection process, thereby providing an efficient, robust, and low-cost solution for heart valve regurgitation detection.

Additionally, some implementations of the present disclosure further relate to a non-transitory computer-readable storage medium storing a computer program. The computer program is executed by the processor to implement the method for detecting heart valve regurgitation.

The method for detecting heart valve regurgitation and the ultrasound system for detecting heart valve regurgitation according to the present disclosure have been described above. However, the structures shown in the above exemplary embodiments are merely one example of the content of the present disclosure, which may be combined with other prior art technologies, and portions of the structures may be omitted or modified without departing from the spirit of the present disclosure.

Furthermore, within the scope of the present disclosure, the embodiments may be freely combined, any structural elements of the embodiments may be modified, or any structural elements of the embodiments may be omitted.

What is claimed is:

1. A method for detecting heart valve regurgitation, comprising:
acquiring a sequence of B-mode ultrasound data to be processed, and a sequence of CF-mode ultrasound data corresponding to the B-mode ultrasound data;
separately performing preprocessing targeting a target region on the sequences of the B-mode ultrasound data and the CF-mode ultrasound data;
performing secondary processing on the preprocessed sequences of the B-mode ultrasound data and the CF-mode ultrasound data, to acquire a valve closure region;
performing regurgitation feature analysis in the valve closure region, to determine a regurgitation frame range; and
extracting target data from the sequence of the CF-mode ultrasound data based on the regurgitation frame range, and generating a regurgitation region identifier.

2. The method according to claim 1, wherein,
the target region is a region of interest comprising a heart valve, and
the preprocessing comprises:
performing detection on each frame of data in the sequence of the B-mode ultrasound data using a target detection algorithm, to acquire position coordinates of the region of interest in each frame of data;
calculating a unified cropping region based on the position coordinates of the region of interest; and
separately performing cropping processing on the sequences of the B-mode ultrasound data and the CF-mode ultrasound data according to the unified cropping region.

3. The method according to claim 1, wherein
the secondary processing comprises dimensional reconstruction, the dimensional reconstruction comprising unfolding the sequence of the B-mode ultrasound data and the sequence of the CF-mode ultrasound data from three-dimensional data to two-dimensional data along a time dimension.

4. The method according to claim 3, wherein
the secondary processing further comprises feature extraction, the feature extraction comprising:
performing trimming on the two-dimensional data for the CF-mode ultrasound data based on blood flow, to obtain a blood flow region range, and performing trimming on the two-dimensional data for the B-mode ultrasound data according to the blood flow region range; and
analyzing a gray-scale intensity distribution of the trimmed two-dimensional data for the B-mode ultrasound data, to determine the valve closure region.

5. The method according to claim 1, wherein
the regurgitation feature analysis comprises:
identifying regurgitation feature points within the valve closure region; and
determining the regurgitation frame range based on distribution characteristics of the regurgitation feature points.

6. The method according to claim 1, wherein
the target data comprises a plurality of regurgitation frames, and
the regurgitation region identifier is generated by:
extracting the regurgitation frames from the sequence of the CF-mode ultrasound data within the regurgitation frame range;
performing segmentation on the regurgitation frames using a segmentation algorithm, to generate the regurgitation region identifier; and
mapping the regurgitation region identifier to a raw ultrasound image.

7. The method according to claim 1, wherein
the sequence of the B-mode ultrasound data and the sequence of the CF-mode ultrasound data represent time sequences of two-dimensional ultrasound images within a same cardiac cycle,
wherein the sequence of the B-mode ultrasound data represents heart valve motion information, and the sequence of the CF-mode ultrasound data represents blood flow information.

8. The method according to claim 1, wherein the regurgitation region identifier is a binary regurgitation mask image.

9. The method according to claim 1,
further comprising performing display enhancement processing on a regurgitation region of an ultrasound image marked by the regurgitation region identifier,
the display enhancement processing comprising:
reducing image visual characteristics of a non-regurgitation region that is not marked by the regurgitation region identifier; and
maintaining velocity attributes of the regurgitation region unchanged.

10. The method according to claim 9, wherein
the image visual characteristics comprise image brightness and image saturation.

11. The method according to claim 1,
further comprising performing highlighted playback processing on regurgitation frames comprised in the target data,
wherein the highlighted playback processing comprises automatically reducing a playback speed of the regurgitation frames during playback.

12. The method according to claim 1,
further comprising automatic measurement processing, the automatic measurement processing comprising:
in a real-time scan mode, automatically positioning a cursor based on the regurgitation region identifier, and automatically initiating an ultrasound measurement mode; and
automatically executing measurement of a parameter related to regurgitation.

13. The method according to claim 12, wherein
the ultrasound measurement mode comprises a continuous wave Doppler mode.

14. An ultrasound system for detecting heart valve regurgitation, comprising:
a memory storing instructions; and
a processor configured to execute the instructions to:
acquire a sequence of B-mode ultrasound data to be processed, and a sequence of CF-mode ultrasound data corresponding to the B-mode ultrasound data;
separately perform preprocessing targeting a target region on the sequences of the B-mode ultrasound data and the CF-mode ultrasound data;
perform secondary processing on the preprocessed sequences of the B-mode ultrasound data and the CF-mode ultrasound data, to acquire a valve closure region;
perform regurgitation feature analysis in the valve closure region, to determine a regurgitation frame range; and
extract target data from the sequence of the CF-mode ultrasound data based on the regurgitation frame range, and generating a regurgitation region identifier.

15. A non-transitory computer-readable storage medium storing thereon instruction that, when executed by a computer, cause the computer to:
acquire a sequence of B-mode ultrasound data to be processed, and a sequence of CF-mode ultrasound data corresponding to the B-mode ultrasound data;
separately perform preprocessing targeting a target region on the sequences of the B-mode ultrasound data and the CF-mode ultrasound data;
perform secondary processing on the preprocessed sequences of the B-mode ultrasound data and the CF-mode ultrasound data, to acquire a valve closure region;
perform regurgitation feature analysis in the valve closure region, to determine a regurgitation frame range; and
extract target data from the sequence of the CF-mode ultrasound data based on the regurgitation frame range, and generating a regurgitation region identifier.
